SEQUENCE LISTING (3 sequences)

SEQ ID NO: 1 - DNA, 36 bp, Artificial Sequence
  Synthetic
aagggagacatctagtgatataagtgtgaactacac

SEQ ID NO: 2 - protein, 147 aa, Homo sapiens
MGHFTEEDKATITSLWGKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTAVASALSSRYH

SEQ ID NO: 3 - protein, 147 aa, Artificial Sequence
  Synthetic
MGHFTEEDKATITSLWDKVNVEDAGGETLGRLLVVYPWTQRFFDSFGNLSSASAIMGNPKVKAHGKKVLTSLGDAIKHLDDLKGTFAQLSELHCDKLHVDPENFKLLGNVLVTVLAIHFGKEFTPEVQASWQKMVTAVASALSSRYH

(19) United States
(12) Patent Application Publication
(10) Pub. No.: US 2022/0243224 A1
(43) Pub. Date: Aug. 4, 2022

(54) VIRAL VECTOR MANUFACTURING METHODS

(71) Applicant: CHILDREN'S HOSPITAL MEDICAL CENTER, Cincinnati, OH (US)

(72) Inventors: Punam MALIK, Cincinnati, OH (US); William SWANEY, Cincinnati, OH (US)

(21) Appl. No.: 17/603,774

(22) PCT Filed: Apr. 15, 2020
(86) PCT No.: PCT/US2020/028277

§ 371 Date: Oct. 14, 2021

Related U.S. Application Data
(60) Provisional application No. 62/833,908, filed on Apr. 15, 2019.

(51) Int. Cl. C12N 15/86; C12N 5/00; C12N 7/00; C12N 5/0789; C07K 14/805

(52) U.S. Cl. CPC C12N 15/86; C12N 5/0087; C12N 7/00; C12N 5/0647; C07K 14/805; C12N 2500/90; C12N 2740/10052; C12N 2830/008; C12N 2830/46; C12N 2830/50; C12N 2500/50; C12N 2740/10043

(57) ABSTRACT

Methods of producing and manufacturing retroviral particles. Such methods may involve the use of an ion-exchange column with an elution buffer comprising one or more salts, wherein the elution buffer has a low total salt concentration (e.g., 400 mM to 800 mM) relative to conventional practice. In some embodiments, the retroviral particles can be generated by host cells transfected with retroviral vectors using polyethylenimine (PEI).

### CROSS-REFERENCE TO RELATED APPLICATIONS

This application claims the benefit of the filing date of U.S. Provisional Application No. 62/833,908, filed on Apr. 15, 2019 under 35 U.S.C. § 119(e). The entire contents of the prior application are incorporated by reference herein.

### GOVERNMENT RIGHTS

This invention was made with government support under HL119810, awarded by the National Institutes of Health. The government has certain rights in the invention.

### BACKGROUND OF THE INVENTION

Retroviral vectors are commonly used in gene therapy due to their capacity to integrate stably into the genome of host cells. Lentiviruses are capable of infecting both dividing and non-dividing cells, giving them the ability to deliver transgenes to cells where other retroviruses may be ineffective.

Retroviral vectors have effectively been used for the delivery and integration of therapeutic transgenes. To date, several genetic diseases have successfully been treated or are in clinical trials using these vectors, for example, sickle cell anemia (SCD), β-thalassemia, X-linked severe combined immunodeficiency (X-SCID), chronic granulomatous disease (CGD), adenine deaminase deficiency (ADA-SCID), and Wiskott-Aldrich syndrome (WAS) (Hacein-Bey-Abina et al., N. Engl. J. Med. 363:355-64, 2010; Hacein-Bey-Abina et al., J. Clin. Invest. 118:3132-42, 2008, Howe et al., J. Clin. Invest. 118:3143-50, 2008, Stein et al., Nat. Med. 16:198-204, 2010, Ott et al., Nature Medicine 12:401-9, 2006, Bortug et al., N. Engl. J. Med. 363:1918-27, 2010).

### SUMMARY OF THE INVENTION

The present disclosure is based, at least in part, on the development of improved methods for preparing retroviral particles (e.g., lentiviral particles), which can be used, for example, in gene therapy. The preparation methods disclosed herein showed production of high quality of retroviral particles, e.g., low defective:infectious ratio, low toxicity, and stable and high gene transfer efficiency both in vitro and in vivo.

Accordingly, one aspect of the present disclosure features a method of producing retroviral particles such as lentiviral particles from a sample by ion-exchange chromatography. The method may comprise, (a) providing a sample containing retroviral particles, (b) loading the sample onto an ion-exchange chromatography column to allow for binding of the retroviral particles to the ion-exchange chromatography column, and (c) eluting the retroviral particles with an elution buffer, which has a salt concentration of about 400 mM to about 800 mM (e.g., about 600 mM) to produce a first solution, which contains enriched retroviral particles. The retroviral particles thus obtained, either directly or after dilution, can be used to infect cells, such as hematopoietic cells.

In some embodiments, the sample to be loaded onto the ion-exchange chromatography column can be a clarified filtrate of the culture medium harvested from an in vitro culture of host cells transfected with a retroviral vector. Such a culture medium may be harvested by a process comprising: (i) transfecting host cells with the retroviral vector, optionally in combination with one or more helper vectors, (ii) culturing the transfected host cells in a first culture medium, and (iii) harvesting the first culture medium 32-48 hours post transfection. In some embodiments, the harvesting step (iii) is only performed once. In some embodiments, the host cells can be initially cultured in a second medium (e.g., when the transfection is performed), which can be replaced with the first medium 4-8 hours post transfection. In some embodiments, the first culture medium is passed through a leukocyte reduction filter (LRF), a 0.45 μM filter, or a combination thereof.

In some embodiments, the retroviral vector used to transfect the host cells may carry a gene of interest, for example, a gene encoding a gamma-globin protein, which may be a human gamma-globin protein. A gene encoding a human gamma-globin protein may comprise one or more intron sequences. In some examples, the human gamma-globin protein can be a wild-type human gamma-globin protein (e.g., comprising the amino acid sequence of SEQ ID NO:1). Alternatively, the human gamma-globin protein may be a mutated human gamma-globin protein as relative to a wild-type counterpart. Such a mutated human gamma-globin may comprise a substitution at a position corresponding to position 17 of SEQ ID NO: 1. In one example, the mutated human gamma-globin may comprise the amino acid sequence of SEQ ID NO: 2.

In some embodiments, the retroviral vector can be a self-inactivating (SIN) retroviral vector. Such a SIN retroviral vector may have a 5′-LTR region and a 3′-LTR region. In some instances, the 3′-LTR may comprise an upstream polyadenylation (polyA) enhancer signal sequence (e.g., an upstream sequence element (USE) derived from an SV40 late polyA signal sequence), one or more copies of a heterologous poly A signal sequence downstream from the 3′ LTR, and/or one or more chromatin insulator elements (e.g., one or more chicken hypersensitive site-4-elements (cHS4s) or the insulator derived from a foamy virus, e.g., those disclosed herein). In some examples, the retroviral vector may contain an erythroid lineage specific enhancer element.

In any of the methods disclosed herein, the transfection of the host cells may be performed in the presence of polyethylenimine (PEI). In some examples, the transfection and culturing of the host cells can be performed in a 10-layer cell stack. Alternatively, cell transfections can be performed in a bioreactor on suspension cells. Alternatively or in addition, the transfection can be performed in the absence of chloroquine, active gassing, or a combination of the two. The cells may be cultured in a medium that has fetal bovine serum (FBS) in a concentration of about 1-6%, or 3%. In some examples, the cells may be cultured in a conditioned medium or in serum free medium.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the instant application, which can be better understood by reference to one or more of these drawings in combination with the detailed description of specific embodiments presented herein.

FIGS. 1A-1C include diagrams showing infectious titers of lentiviral particles obtained from different transfection methods. FIG. 1A: a schematic illustration of an exemplary lentiviral vector carrying a gamma-globin gene. FIG. 1B: a chart showing infectious titers (infectious lentiviral particles/mL) of unconcentrated lentiviral particles obtained from calcium phosphate-mediated transfection and PEI-mediated transfection. FIG. 1C: a chart showing infectious titers (infectious lentiviral particles/mL) of concentrated lentiviral particles obtained from calcium phosphate-mediated transfection and PEI-mediated transfection.

FIGS. 2A-2B include diagrams showing quality of lentiviral particles in a first harvest of culture supernatant and in a subsequent (second) harvest of culture supernatant. FIG. 2A:

a chart showing defective:infectious ratio of lentiviral particles in 1^{st} and 2^{nd} harvest samples. FIG. 2B: a chart showing toxicity of 1^{st} and 2^{nd} harvest samples to CD34+ cells (viable CD34+ cell counts) when concentrated vector particles were added in increasing amounts (increasing multiplicity of infection (MOI)).

FIGS. 3A-3B include diagrams showing quality of lentiviral particles recovered from ion-exchange chromatograph by low salt elution and high salt elution. FIG. 3A: a chart showing defective:infectious ratio of lentiviral particles recovered from low salt elution (new) and high salt elution (old). FIG. 3B: a chart showing gene transfer efficiency of lentiviral particles recovered from low salt elution and high salt elution at low MOI and high MOI in CD34+ cells. Gene transfer efficiency is graphed as vector copy number per cell (VCN) determined at 2 weeks in culture after the lentiviral gene transfer into CD34⁺ cells.

FIG. 4 is a chart showing transducibility of lentiviral vectors recovered from ion-exchange chromatography with low salt elution or high salt elution using samples of 1st Harvest or a pool of 1st and second Harvests. CD34+ cells were transduced at increasing MOI of concentrated lentiviral vector produced using a high salt elution (Traditional Elution Method; 2 Harvests), low salt elution with two harvests/collections (New Elution Method; 2 Harvests) or low salt elution with a single harvest/collection (New Elution Method; 1 Harvest).

FIG. 5 is a diagram illustrating an exemplary experimental overview for studying in vivo activity of lentiviral vectors (particles) produced by the manufacturing method disclosed herein, which involves PEI transfection, low salt elution, and one harvest.

FIGS. 6A-6B include diagrams showing that viral vectors produced by the manufacturing method disclosed herein did not show toxicity to human CD34⁺ hematopoietic stem and progenitor cells (HSPC) in vitro. FIG. 6A: a diagram showing total cell count at day 3 after transfection as determined by a hemocytometer. FIG. 6B: a diagram showing cell viability at day 3 after transfection determined by flow cytometry measuring green fluorescence.

FIGS. 7A-7B include diagrams showing that viral vectors produced by the manufacturing method disclosed herein showed increased vector copy numbers in CD34⁺ HSPC cells with increasing vector MOI, validating high quality vector preparation. FIG. 7A:

a diagram showing bulk culture vector copy number in a 14-day liquid culture. FIG. 7B: a diagram showing CFUc vector copy number at 12-14 days of culture.

FIG. 8A-8B include diagrams showing that the vector copy number (VCN) is stable and maintained in vivo, including in whole bone marrow and in human CD34⁺ HSPC isolated from bone marrow of immune-deficiency mice 4-5 months post-transplant. FIG. 8A: a diagram showing vector copy number in bone marrow from the whole bone marrow of individual mice. FIG. 8B: a diagram showing vector copy number in bulk culture of human CD34+ cells after isolation from mice post-transplant (groups were pooled).

### DETAILED DESCRIPTION OF THE INVENTION

Retroviral vectors such as lentiviral vectors and gamma retroviral vectors provide an efficient means for introducing genetic modifications, such as introducing new genes, into human and animal cells. Various generations of retroviral vector systems have been developed to minimize the safety considerations due to the pathogenicity of HIV-1. Third-generation, self-inactivating retroviral vectors have been used in clinical trials for introducing genes into host cells such as hematopoietic for treating genetic disorders and hemoglobinopathies.

The retroviral vectors described herein may comprise the viral elements such as those described herein from one or more suitable retroviruses, which are RNA viruses with a single strand positive-sense RNA molecule. Retroviruses comprise a reverse transcriptase enzyme and an integrase enzyme. Upon entry into a target cell, retroviruses utilize their reverse transcriptase to transcribe their RNA molecule into a DNA molecule. Subsequently, the integrase enzyme is used to integrate the DNA molecule into the host cell genome. Upon integration into the host cell genome, the sequence from the retrovirus is referred to as a provirus (e.g., proviral sequence or provirus sequence). This efficient gene transfer mechanism has made retroviral vectors highly valuable tools in gene therapy, because they can be used for long term transgene expression in host cells.

The present disclosure provides improved methods for preparing or manufacturing retroviral particles, which can be used to infect host cells of interest. The methods disclosed herein may have one or more of the following features, which led to production of higher infectious retroviral particles as compared with conventional procedures.

First, the preparation/manufacture methods disclosed herein may involve the use of PEI for transfecting a retroviral vector to host cells, as opposed to calcium phosphate, which is commonly used in conventional viral vector transfection processes. Without being bound by theory, calcium phosphate was found to be negatively influenced by small pH changes of HEPES-buffered saline used in the transfection procedure. Results reported herein show that, unexpectedly, the use of PEI in the transfection procedure obviated these concerns and led to more reproducible procedures. Moreover, the preparation or manufacture methods disclosed herein may not use chloroquine during cell transfection and/or cell culture. These features were found not to improve the titers of retroviral particles. Removal of such could eliminate unnecessary materials and decrease the overall number of total open manipulations performed during manufacturing.

Alternatively or in addition, the methods disclosed herein may involve a single collection/harvest of supernatant from transfected cells, which improves retroviral particle quality, for example, improved infectious: defective virus ratio, prior to the further purification and centration via, e.g., ion-exchange chromatography.

Alternatively or in addition, the preparation or manufacture methods disclosed herein involve ion-exchange chromatography for enriching retroviral particles using an elution buffer having a low salt concentration (e.g., about 400-800 mM), as opposed to the high salt concentration (e.g., 1200 mM) used in conventional methods. The low salt concentration leads to production of retroviral particles having a higher infectious capacity and prevents tonicity stress induced by lowering the high salt concentration to a lower level prior to infection.

### Transfection of Host Cells with Retroviral Vectors

The retroviral vectors disclosed herein comprise one or more elements derived from a retroviral genome (naturally-occurring or modified) of a suitable species.

Retroviruses include 7 families: alpharetrovirus (Avian leucosis virus), betaretrovirus (Mouse mammary tumor virus), gammaretrovirus (Murine leukemia virus), deltaretrovirus (Bovine leukemia virus), epsilonretrovirus (Walleye dermal sarcoma virus), Lentivirus (Human immunodeficiency virus 1), and spumavirus (Human spumavirus). Six additional examples of retroviruses are provided in U.S. Pat. No. 7,901,671.

Lentivirus is a genus of retroviruses that in nature give rise to slowly developing disease due to their ability to incorporate into a host genome. Modified lentiviral genomes are useful as viral vectors for the delivery of a gene to a host cell. Host cells can be transfected with lentiviral vectors, and optionally additional vectors for expressing lentiviral packaging proteins (e.g., VSV-G, Rev, and Gag/Pol) to produce lentiviral particles in the culture medium.

### (i) Retroviral Vectors

Viral elements, such as those described herein, from a suitable retrovirus can be used to construct the retroviral vectors described herein. Non-limiting examples of retroviral vectors include human immunodeficiency viral (HIV) vector, avian leucosis viral (ALV) vector, murine leukemia viral (MLV) vector, murine mammary tumor viral (MMTV) vector, murine stem cell virus, and human T-cell leukemia viral (HTLV) vector. These retroviral vectors comprise proviral sequences from the corresponding retrovirus.

The retroviral vectors such as lentiviral vector disclosed herein may comprise a 5′ lentiviral long terminal repeat (5′ LTR) and a 3′ lentiviral long terminal repeat (3′ LTR). The 5′ LTR and/or 3′ LTR can be the native 5′ LTR and native 3′ LTR of a lentiviral genome. Alternatively, either one may be modified, e.g., including deletions, insertions, and/or mutations relative to the native sequences. In some examples, the 3′-LTR may further comprise a polyadenylation (poly A) enhancer signal sequence, which is located upstream of the cleavage/polyadenylation (polyA) site (e.g., AAUAAA) and function to increase the polyA site efficiency and thus polyadenylation efficiency. Exemplary polyadenylation enhancer signal sequence includes upstream sequence element (USE) from a suitable viral gene, for example, simian virus 40 (SV40) late gene. Inclusion of such a polyA enhancer signal sequence may facilitate transcription termination and reduce read-through of vector transcript and improving packaging efficiency, which would lead to increased viral titer.

In some instances, the lentiviral vector disclosed herein can be a self-inactivating (SIN) vector, which may contain a deletion in the 3′ long terminal repeat region (LTR). In some examples, the vector may contain a deletion within the viral promoter.

In addition to the LTRs described herein, the retroviral vectors also comprise components necessary for the basic functionality of the retroviral vector, for example, capable of being replicated, packed into viral particles, and/or capable of drive expression of genes of interest carried thereby in host cells. Such essential elements for constructing retroviral vectors are well known to those skilled in the art.

In some embodiments, the retroviral vectors described herein may comprise one or more of the following components: (i) a psi (ψ) packaging signal; (ii) a rev response element (RRE); (iii) a gag element; (iv) an env splice acceptor sequence; (v) one or more copies of a heterologous polyA signal sequence downstream from the 3′ LTR; (vi) one or more chromatin insulator elements; (vii) a central polypurine tract (cPPT); and (viii) a post-transcriptional regulatory element (PRE).

A psi (ψ) packaging signal, also known as an encapsidation sequence, regulates the packaging of retroviral RNA into viral capsids during replication. It is typically placed downstream of 5′ long terminal repeat in a retroviral vector to effectively package and deliver transgene carried by the retroviral vector.

A rev response element (RRE) is a domain located in the env region. A RRE may have up to 360 nucleotides long within the ‘env gene’. Rev protein binds to the RRE to regulate the expression of viral genes. The Rev/RRE system facilitates nuclear export of mRNAs.

A gag (group-specific antigen) element encodes for the structural proteins (or a portion thereof) of a retrovirus, i.e., matrix, capsid and nucleocapsid components. In some instances, the retroviral vector described herein may contain a gag fragment that is the 5′ fragment of a gag gene. Such a fragment may contain 250-650 bps (e.g., about 360 bps or 600 bps). Containing such a short gag fragment may enhance viral titer of retroviral vectors carrying a large gene of interest (for example, a globin gene). See, e.g., US20150316511, the relevant disclosures are incorporated by reference herein. In other instances, the retroviral vector described herein may be free of any gag fragment.

An env splice acceptor sequence is a nucleotide sequence near the 3′ end of the pol coding region in a retroviral genome. The splice acceptor sequence regulates the splicing of transcripts. It also enables the expression of the env coding region.

In some instances, the retroviral vector may comprise one or more heterologous polyA signaling sites, which may be located downstream from the 3′ LTR. Such heterologous polyA signaling sites may not be of a viral origin (e.g., from a non-viral gene such as a β-globin gene). Alternatively, the heterologous polyA signaling sites may be derived from a viral gene which is from a different viral species as the retroviral vector that contains the heterologous polyA signaling sites. Inclusion of such heterologous polyA signaling sites may enhance polyadenylation efficiency, thereby further reducing read-through of vector transcript and improving packaging efficiency, which would lead to increased viral titer.

In some embodiments, the retroviral vector may include one or more chromatin insulator elements. Chromatin insulators are promoter or enhancer sequences that resist heterochromatin formation. In some embodiments, a chromatin insulator can be a fragment of about 1 kb in length that blocks transcriptional activation by enhancers. It may function as barrier elements, as described herein to, inter alia, prevent the spread of heterochromatin and silencing of genes, reduce chromatin position effects and have enhancer blocking activity. These properties are desirable for consistent predictable expression and safe transgene delivery with randomly integrating vectors. Insulated vectors have reduced chromatin position effects and, provide consistent, and therefore improved overall expression.

In some examples, the one or more chromatin insulator elements in the retroviral vector described herein may be chicken hypersensitive site-4 elements (cHS4), which is a chromatin insulator from the chicken β-globin locus control region. Arumugam et al., PLoS ONE 4(9): e6995, 2009. In some instances, one or more full-length chromatin insulators (about 1.2 kb) of hypersensitive site-4 (cHS4) from the chicken p-globin locus can be inserted in the 3′LTR to allow its duplication into the 5′LTR in retroviral vectors such as gamma retrovirus or Lentivirus. In other instances, a truncated cHS4 fragment comprising a ˜250-bp core may be used in the retroviral vector described herein. Such a core fragment may 3 0 be combined with a 3′ ˜400-bp fragment from the cHS4 element. In one example, a functional reduced-length insulator of about 650 base pairs, including the core sequence and the 3′-fragment, can be used in constructing the retroviral vector described herein. Such cHS4-derived insulator sequences are described in US 20150316511, the relevant disclosures are incorporated by reference herein.

Non-limiting examples of other chromatin insulators include ArsI (derived from the sea urchin arylsulfatase gene locus), sns5 (derived from the sea urchin H2A early histone gene), Ankyrin-1 gene promoter element, Drosophila gypsy element (Emery, Human Gene Therapy 22(6):761-74, 2011).

The foamy virus insulator, a 36-bp sequence (SEQ ID NO:3 AAGGGAGACATCTAGTGATATAAGTGTGAACTACAC) found in the LTRs of foamy virus vectors that has potent insulator activity (Goodman, J. Virology 2017). A central polypurine tract (cPPT) directs penetration of viral particles through the nuclear membrane. In retroviral replication, it functions as a primer for synthesis of plus-strand DNA. It has been shown to increase the transduction efficiency and transgene expression when incorporated into retroviral vectors.

A post-transcriptional regulatory element (PRE) is a sequence that, when transcribed, enhances the expression of a transgene in a viral vector. It has been shown to increase the transduction efficiency and transgene expression when incorporated into retroviral vectors.

In some embodiments, the PRE used in the retroviral vector is a PRE from a Hepatitis B virus (HPRE) or a PRE from a Woodchuck Hepatitis virus (WPRE). In some embodiments, there is more than one PRE in the retroviral vector, and the more than one PRE can be HPRE, WPRE, or a mixture thereof. In one embodiment, the retroviral vector does not include a PRE.

The retroviral vectors described herein may further comprise additional functional elements as known in the art to address safety concerns and/or to improve vector functions, such as packaging efficiency and/or viral titer. Additional information may be found in US20150316511 and WO2015/117027, the relevant disclosures of each of which are herein incorporated by reference for the purpose and subject matter referenced herein.

Additional information for lentiviral vectors can be found in, e.g., WO2019/056015, the relevant disclosures of which are incorporated by reference herein for this particular purpose.

Any of the retroviral vectors may further comprise a gene of interest. In some instances, the gene of interest encodes a therapeutic agent such as a therapeutic protein or therapeutic nucleic acid. Expression of the therapeutic agent may be under the control of a suitable promoter in operable linkage to the gene of interest. Exemplary therapeutic proteins include antibodies, growth factors, cytokines, coagulation factors, enzymes, or hemoglobins.

In one particular example, the gene of interest may encode a gamma globin, for example, a human gamma globin. The human gamma globin may be a wild-type human gamma globin. Alternatively, the human gamma globin may be a mutated form, which may have higher tendency to form HbF as compared with the wild-type counterpart. Such a mutant may contain an amino acid residue variation at position 17 of a wild-type human gamma-globin. Exemplary amino acid sequences of wild-type and mutant human gamma-globin proteins are provided below:

Amino acid sequence of a wild-type human γ-globin protein:

(SEQ ID NO: 1)
MGHFTEEDKATITSLWGKVNVEDAGGETLGRLLVV
YPWTQRFFDSFGNLSSASAIMGNPKVKAHGKKVLT
SLGDAIKHLDDLKGTFAQLSELHCDKLHVDPENFK
LLGNVLVTVLAIHFGKEFTPEVQASWQKMVTAVAS
ALSSRYH

Amino acid sequence of a mutant human γ-globin protein (substitution in boldface and underlined):

(SEQ ID NO: 2)
MGHFTEEDKATITSLW [symbol] KVNVEDAGGETLGRLLVV
YPWTQRFFDSFGNLSSASAIMGNPKVKAHGKKVLT
SLGDAIKHLDDLKGTFAQLSELHCDKLHVDPENFK
LLGNVLVTVLAIHFGKEFTPEVQASWQKMVTAVAS
ALSSRYH

### (ii) Transfection of Host Cells

Any of the retroviral vectors as disclosed herein can be introduced into suitable host cells permissive for production of retroviral particles. Examples include, but are not limited to, 293T cells, 293FT cells, COS cells, L cells, 3T3 cells, and Chinese hamster ovary (CHO) cells. In some instances, the retroviral vectors lack one or more retroviral packaging proteins (e.g., those noted above). Such retroviral vectors can be co-transfected with one or more additional vectors capable of expressing the retroviral packaging proteins. For, a retroviral vector carrying a gene of interest (e.g., coding for a human gamma globin as disclosed herein) may be co-transfected with one or more helper vectors, which are designed for expressing viral proteins necessary for viral genome replication and/or viral particle packaging, e.g., VSV-G proteins, Rev protein, gag/pol proteins, or a combination thereof. In some examples, a retroviral vector carrying a gene of interest (e.g., coding for a human gamma globin as disclosed herein) may be co-transfected with three additional helper vectors, each being designed for expressing VSV-G protein, Rev protein, and gag/pol proteins.

Alternatively, retroviral packaging cells can be used as the host cell. Such cells stably express retroviral proteins essential for viral particle packaging. Any of the retroviral vectors can be introduced into retroviral packaging cells in the absence of other vectors for expressing packaging proteins.

Methods for transfecting viral vectors into host cells are well known in the art. Some transfection approaches are chemical, e.g., using liposomes or calcium phosphate. Others may be non-chemical, e.g., electroporation or optical transfection. In preferred examples, the methods disclosed herein may involve the use of PEI for transfection. It was reported herein that the use of PEI in the transfection process has obviated negative impacts resulting from other conventional approaches, such as using calcium phosphate. For example, use of PEI for transfection is unlikely to be influenced by small pH changes of the buffer solution used to dissolve viral vectors and the transfection agent (e.g., PEI or calcium phosphate). It also can minimize changes of phosphate concentration in the culture medium, which can be critical to cell growth, and minimize complex formation during incubation, which is a common concern of using calcium phosphate.

To transfect suitable host cells with any of the retroviral vectors disclosed herein, the cells can be seeded in a suitable container. Suitable containers include petri dishes, flasks, vials, multi-tray systems (Cell Factories, Cell Stacks), or similar containers suitable for cell culture. Many varieties are known in the art and are commercially available, for example Cell BIND™ plates and flasks from Corning™. When needed, the surface of the container can be pretreating with chemicals such as Poly-L-Lysine to increase cell adherence. Alternatively, the surface of the container may not be pretreated.

In some examples, the methods disclosed herein may use Cell Bind™ 10 layer cell stacks for cell growth and transfection. Using this type of containers can decrease the total number of stacks needed for manufacturing, and also eliminates the need to Poly-L-Lysine treat the stacks. This reduces the total number of open manipulations performed during the aseptic manufacturing procedure and eliminates the use of an unnecessary solution during manufacture.

The host cells can be cultured in the container for a suitable period in a suitable medium (complete medium) to allow growth of the cells to appropriate confluency and conditions for transfection. Complete medium is a term known in the art as referring to a medium for an in vitro culture that contains supplemental nutrients as well as basic nutrients to support cell growth requirements. For manufacturing purposes (for producing a large quantity of retroviral particles), suspension cell culture may be used. Medium selection is often dependent on the host cells used. Culture media are widely available and known in the art, but will contain a carbon source, water, various salts, amino acids and nitrogen, along with other nutrients or growth factors specifically tailored to the host cells. Exemplary culture media include Dulbecco's Modified Eagle's Medium (DMEM) or a serum free medium. Additionally, culture medium may be modified to suit the needs of the host cells used in the methods described herein, for example, by the addition of other components such as FBS, sodium-pyruvate, benzonase, magnesium chloride, chloroquine, a transfection agent or compound, or a combination thereof.

In some examples, the culture medium used herein can be DMEM, which may be supplemented with FBS (e.g., 8 to 12% such as 10%), sodium-pyruvate (e.g., 0.5 to 3% such as 1%) or a combination thereof. Alternatively, the culture medium may be a serum free medium.

In some instances, the host cells are cultured and/or transfected in the absence of chloroquine, as opposed to conventional approaches. It was found that the use of chloroquine in the cell stacks did not improve the titers of retroviral particles. Their removal also eliminates un-necessary materials as well as decreases the overall number of total open manipulations performed throughout the manufacturing process.

When the host cells are in condition for transfection, a mixture containing a retroviral vector (e.g., those disclosed herein), the additional vectors if any, transfection agent, and the culture medium can be prepared and incubated for a suitable period. A transfection agent is a substance that facilitates entry of the DNAs into the host cells. Many transfection agents are known in the art and widely available. Examples include calcium phosphate, highly branched organic compounds, cationic polymers (such as PEI), and liposomes. In a preferred example, the method disclosed herein involves the use of PEI as the transfection agent.

The mixture containing the DNAs and transfection agent can then be added to the cell culture to allow for delivery of the retroviral vector and any additional vectors into the host cells. In some embodiments, the method disclosed herein involves incubating the transfection mixture with host cells growing in suspension. After a suitable period, the whole content, including the host cells and the transfection mixture, may be placed in a culture containing having multiple stacks (e.g., 5-30 such as 10-stacks) or in large bioreactors. The host cells may be adherent cells or suspension cells. The host cells and the transfection mixture may be incubated in the presence of chloroquine. In a preferred embodiment, the host cells and the transfection mixture may be incubated in the absence of chloroquine for the benefits noted herein.

After transfection, the cells may be cultured for a suitable period, e.g., 4-24 hours, such as 4-6 hours or 12-18 hours. The culture medium can then be replaced with a second culture medium. In some instances, the second culture medium can be a complete medium, which may comprise DMEM supplemented with FBS (e.g., about 2-5% such as 3%), or a serum free medium, 1% sodium pyruvate (e.g., about 0.5-3% such as 1%), benzonase (about 30-80 U/mL such as 50 U/ml), and one or more salts such as MgCl₂. In some embodiments, the methods disclosed herein do not use a conditioned medium in the medium change post transfection. Conditioned medium was found not to be required for high titer retroviral particle manufacture. Its removal eliminates additional storage requirements, eliminates the need to mix fresh media and conditioned media, and improves harvest media quality as both the pH and amount of glucose present in the mixture of conditioned medium and fresh medium (typically complete culture media) was significantly lower than that in non-mixed complete culture media.

Alternatively, the second culture medium can be a mixture of conditioned medium and fresh medium containing about 8-12% FBS (e.g., 10%) at a suitable ratio, for example,1:1, 2:1, 3:1, 4:1, 1:2, 1:3, 1:4. In one particular example, the ratio can be 1:1. Conditioned medium refer to spent media harvested from cultured cells. They contain metabolites, growth factors, and extracellular matrix proteins secreted into the media by the cultured cells. The fresh medium can be any of the culture medium disclosed here, for example, a complete culture medium, which can be DMEM (e.g., high glucose; 4,500 mg/L) in GlutaMAX-I and HEPES buffer supplemented with 10% FBS and 1 mM sodium pyruvate.

The transfected cells can be further cultured for a suitable period, e.g., 12 hours to 48 hours (e.g., around 15 to 18 hours). The culture supernatant can then be collected. Such supernatant contains retroviral particles for further enrichment. In some embodiments, the supernatant can be collected about 28-48 hours (e.g., 32-42 hours) post transfection only once (i.e., a single collection), as opposed to second collection/harvest adopted in conventional approaches 15-24 hours later. The elimination of a second collection/harvest of supernatant was found to improve retroviral particle quality (e.g., improve the infectious: defective virus ratio) prior to further purification and concentration. It was found that the second harvest has less number of infectious particles compared to the first harvest, and contains more non-infectious particles. See Examples below. Hence higher quality vector is generated initially (harvest 1), and mixing harvest 1 and 2 may increase the defective:infectious particle ratio in the retroviral particle preparation. The timing of the single collection/harvest was optimized to get the best ratio of infectious:defective particles within the single collection/harvest.

The supernatant collection can then be passed through a LRF, followed by a 0.45 μM filter. The passing through solution, which contains viral particles, can be subject to further purification and concentration by, e.g., ion-exchange chromatography.

### Retroviral Particle Purification Via Ion-Exchange Chromatography

The retroviral particle-containing solutions disclosed herein can be subject to ion-exchange chromatography to enrich the retroviral particles. Since retroviral particles are negatively charged on their surface, membranes or resins having positively charged surfaces are typically used in ion-exchange chromatography for enriching retroviral particles. Exemplary ion-exchange membranes or columns for use in the methods disclosed herein include diethylaminoethyl cellulose (DEAE-C), Mustang-Q™ column, quaternary ammonium cation resins (Q), triethylaminoethyl (TEAE), diethyl-2-hydroxypropylaminoethyl (QAE), sulpho (S), sulphomethyl (SM), sulphopropyl (SP), carboxy (C), and carboxymethyl (CM).

The ion-exchange process used in the preparation and manufacturing methods disclosure herein may use an elution buffer having a low salt concentration, as opposed to eluting buffers having a high salt concentration (e.g., 1200 mM) used in conventional methods. A buffer having a low salt concentration as used herein refers to a buffer having a total salt concentration less than 1000 mM. In some embodiments, the salt concentration of the elution buffer used in the methods disclosed herein may range from about 400 to about 800 mM. In one particular example, the salt concentration can be around 600 mM.

The use of low salt, e.g., 600 mM, was found to improve the quality of retroviral particles eluted. For example, retroviral particles eluted with 600 mM salt had a higher infectious:defective retroviral particle ratio, as determined by infectious titers and p24 ELISA and was sufficient to remove the bound retroviral particles from the column. See Examples below. Furthermore, Lentivirus is salt sensitive. As such, the use of low salt concentration for elution puts the eluted virus particles into a lower salt concentration during the remainder of retroviral particle processing and can prevent the tonicity stress induced by lowering the salt concentration from 1200 mM to 400 mM as used in conventional methods.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The low salt elution buffer for use in the present disclosure may contain one or more suitable salts, which can be those commonly used in pertinent art. Examples include calcium chloride, magnesium chloride, sodium chloride, potassium chloride, and ammonia chloride. The elution buffer may further comprise a buffering agent, which can be any weak acid or base capable of maintaining the acidity (pH) of a solution bear a chosen value after the addition of another acid or base. Examples include TAPS ([Tris(hydroxymethyOmethylamino]propanesulfonic acid), Bicine (2-(Bis(2-hydroxyethyl)amino)acetic acid), Tris (Tris(hydroxymethyl)aminomethane) or, (2-Amino-2-(hydroxymethyl)propane-1,3-diol), TAPSO (3-[N-Tris(hydroxymethyOmethylamino]-2-hydroxypropanesulfonic acid), HEPES (4-(2-hydroxyethyl)-1-piperazineethanesulfonic acid), TES (2-[[1,3-dihydroxy-2-(hydroxymethyl)propan-2-yl]amino]ethanesulfonic acid), MOPS (3-(N-morpholino)propanesulfonic acid), PIPES (Piperazine-N,N′-bis(2-ethanesulfonic acid)), Cacodylate (Dimethylarsenic acid), and MES (2-(N-morpholino)ethanesulfonic acid). In one example, the buffering agent is Tris-HCl (e.g., at about 20-40 mM, such as 25 mM). The elution buffer may have a pH ranging from about 7.5-8.5 (e.g., 8).

To enrich retroviral particles using ion-exchange chromatography, a suitable ion-exchange membrane or column as those disclosed herein can be washed and balanced using a suitable buffer. A solution containing retroviral particles (e.g., cell culture supernatant collection as disclosed herein) can be loaded onto the membrane or the column under conditions allowing for binding of the viral particles on the membrane or column. When needed, the retroviral particle-loaded membrane or column can be washed one or more times to remove impurities attached to the membrane or column. The bound retroviral particles can then be eluted using the low salt elution buffer disclosed herein.

In some embodiments, the eluted fraction containing retroviral particles can be diluted (e.g., immediately) to further reduce the salt concentration in the retroviral particle-containing solution. For example, the dilution may be performed by a 1:1 mixing of the elution fraction with water so as to reduce the salt concentration by 50% (e.g., to 300 mM). In some embodiments, this dilution step can be performed prior to concentration via tangential flow filtration and diafiltration.

The retroviral particles thus prepared can be used to infect cells for therapeutic or research purposes. In some instances, the retroviral particles, carrying a gene of interest that encodes a therapeutic agent, can be used to infect target host cells (e.g., human cells) for treating target diseases. For example, the retroviral particles, carrying a gene coding for a human gamma-globin, can be used to infect hematopoietic cells (HCs), which may be any cells having hematopoietic origin. HCs include those lodged within the bone marrow (e.g., HSCs), cells differentiated therefrom (for example, those circulating in the blood such as red blood cells, white blood cells, and platelets), and HSCs derived from in vitro differentiation of stem cells (e.g., induced pluripotent stem cells or iPSCs). The infected HCs are capable of expressing the therapeutic agent carried by the retroviral particles (e.g., human gamma-globin proteins as those disclosed herein) and can be used in hematopoietic cell transplantation for treating diseases such as hemoglobinopathy. Hemoglobinopathy refers to a disorder associated with a genetic defect that results in abnormal structure of one of the globin polypeptide of hemoglobin or reduction of the globin polypeptide, e.g., alpha- (α-), beta- (β-), or gamma- (γ-) globin. Common hemoglobinopathies include sickle-cell disease and thalassemia such as β-thalassemia. Additional information for using Lentivirus-mediated HS transplantation for treating such hemoglobinopathy can be found in International Patent Application No. PCT/US18/58790, the relevant disclosures of which are incorporated by reference for this specific purpose.

The method of production can be used in general for all retroviral vectors (non-exclusive examples being Lentivirus, gamma retrovirus, and foamy virus vectors).

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D.N. Glover ed. 1985); Nucleic Acid Hybridization (B. D. Hames & S. J. Higgins eds.(1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Viral Vector Transfection Using Polyethylenimine Yielded High Quality Lentiviral Particles as Compared with Transfection Using Calcium Phosphate

In calcium phosphate-mediated transfection, DNA co-precipitates with Ca₃(PO₄)₂ and adheres onto the cell membrane. The DNA can then be taken up into the cell via endocytosis. This approach is inexpensive and effective but can be very pH sensitive and complexion time dependent. In PEI-mediated transfection, PEI condenses DNA into positively charged particles, which can bind to the anionic cell surface and subsequently be taken up by the cells via endocytosis. This approach is relatively expensive as compared with the calcium phosphate approach and can induce cytotoxic at higher doses. On the other hand, it is not as pH sensitive and complexion time dependent as the calcium phosphate approach.

This Example compares the yields and quality of lentiviral particles produced by transfecting host cells with lentiviral vectors via the calcium phosphate approach and the PEI approach.

### PEI-Mediated Transfection

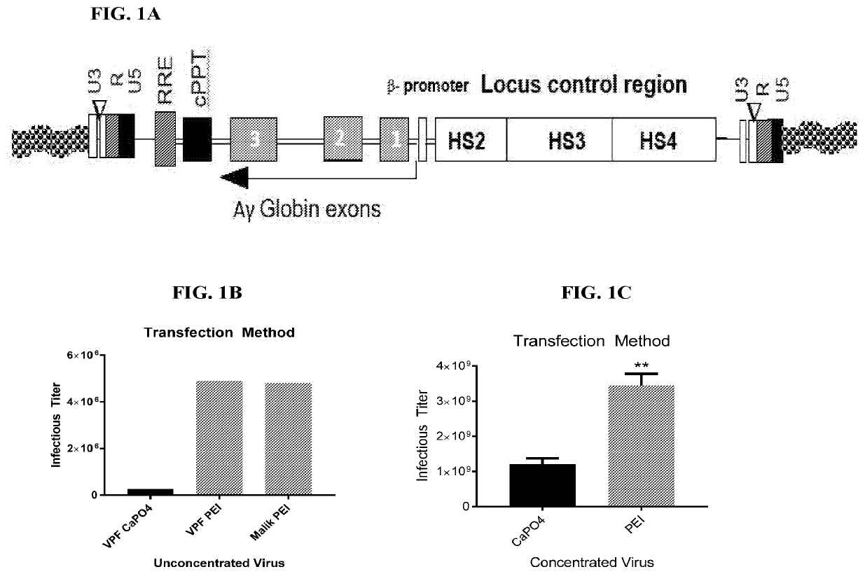

Three days prior to pre-seeding cells, thirty 10-layer Cell Bind™ cell stacks were placed inside an incubator to allow equilibration of gas mixtures in the cell stacks with that of the incubator. The cell stacks were pre-seeded with HEK 293T host cells the day prior to transient transfection at a density of 1.38×10⁵ viable cells/cm². The following day, the host cells were transiently transfected using PEI and the sGbG^{M} lentiviral vector (structure shown in FIG. 1A), together with three helper vectors for expressing VSV-G, Gag/pol, and REV. The sGbG^{M} lentiviral vector encodes the human gamma globin mutant of SEQ ID NO:2. Five to six hours post transfection, the cell culture medium (containing DMEM supplemented with 10% FBS, 1% sodium-pyruvate, and the PEI transfection mix) was removed from each stack and replaced with complete culture medium (DMEM supplemented with 3% FBS, 1% sodium-pyruvate, benzonase (50 U/mL), and MgCl₂). The 10-layer cell culture stacks incubated in an incubator, which had been pre-warmed to 37° C. overnight. The culture supernatant was collected 32-42 hours post transfection for further processing.

The PEI approach was optimized with respect to supernatant collection timing and addition of benzonase (the Vector Production Facility, Cincinnati Children's Hospital (VPF) approach and an approach developed by Punam Malik's Laboratory, Cincinnati Children's Hospital (Malik)). See FIG. 1B.

### Calcium Phosphate-Mediated Transfection

Transient Transfection was performed using calcium phosphate as follows. Briefly, forty 5-layer cell stacks are pre-treated with Poly-L-Lysine and stored at ambient until filled. Cells were harvested and the used media collected and stored at 2-8° C. until use at media change. A quantity of cells sufficient to seed a 5-layer cell stack at 2.67×10⁵ viable cells/cm² was placed into each of forty 1L storage bottles. Complete culture media (DMEM with 10% FBS, and 1% Na-Pyruvate) was then added to each bottle to a final volume of 700 mL. A calcium phosphate transient transfection mixture containing the lentiviral vector and the three helper vectors noted above was prepared. After a 20 minute incubation, 42 mL of the transfection mixture was added to each bottle containing cells along with 750 μL of chloroquine (25 mM final concentration). The contents of each bottle (cells, complete culture media, transfection mixture, and chloroquine) was then added to each 5-layer stack. A 5% CO₂/40% O₂ gas mixture was blown into each 5-layer stack through a 0.2 μM sterile filter and the stacks incubated. Approximately 16 hours post transfection, the media was changed to a 1:1 mixture of conditioned medium and fresh medium containing 10% FBS and the stacks returned to the incubator. Approximately 6 hours post media change, each stack was removed from the incubator, sterile filtered benzonase (50 U/mL) and MgCl₂ were added to each stack. A 5% CO₂/40% O₂ gas mixture was blown into each 5-layer stack through a 0.2 sterile filter as before and the stacks incubated. The culture supernatant was then collected for further analysis.

Concentrated and unconcentrated lentiviral particles produced from the PEI and calcium approaches disclosed above were titered on murine erythroleukemia (MEL) cells. Infectious titers were determined based on the proportion of HbF expressing MEL cells from the different serial dilutions of lentiviral particles. As shown in FIG. 1B and FIG. 1C, the infectious titers of both unconcentrated (FIG. 1B) and concentrated (FIG. 1C) of lentiviral particles produced by the PEI transient transfection method are substantially greater than those produced by the calcium-phosphate transient transfection. The VPF and Malik PEI approaches produced comparable viral infectious titers. Both approaches were superior to the calcium phosphate transfection method.

### Example 2: Single Harvest Increases Ratio of Infectious Lentiviral Particles

In conventional methods, virus-containing cell culture supernatant is typically harvested twice, one at 24 hours after transfection and the other at ˜48 hours after transfection, as cells continue to produce virus. Two collections helps rejuvenate the medium and keep transfected cells healthy. However, highest amount of virus typically is produced in the first 24 hours post transfection. After that, virus is produced at a declining rate and the second harvest only contains 60-80% of infectious viral particles as compared to the first harvest. This Example compares the virus quality, represented by the defective:infectious ratio, of a single collection and of two collections.

For the single collection approach, a single collection of the Lentivirus-containing cell culture supernatant was collected between thirty-two and forty-two hours post transfection. The supernatant was passed through a leukocyte reduction filter (LRF), and then 0.45 μm filtered.

For the two collection approach, the lentiviral particle-containing cell culture supernatant was first collected approximately fifteen to eighteen hours post transfection (1^{st} Harvest), passed through a LRF and stored at 2-8° C. for approximately 24 hours. Each cell culture container was then refed with 750 mL of complete culture media and incubated an additional 24 hours. After 24 hours, the lentiviral particle-containing cell culture supernatant was collected a second time (2^{nd} Harvest), passed through a LRF and then combined with the

LRF filtered Pt collection. The 60 L pool of LRF filtered 1^{st} and 2^{nd} collections was then 0.45 μm filtered.

The total viral particles in the supernatant samples were determined using a p24 ELISA. Not all virus particles are infectious (i.e., able to transduce cells, as many are defective particles or empty particles). Transducing/Infectious particles were determined from transgene (here the gamma-globin gene) expressing cells transduced at a serial dilution of vector.

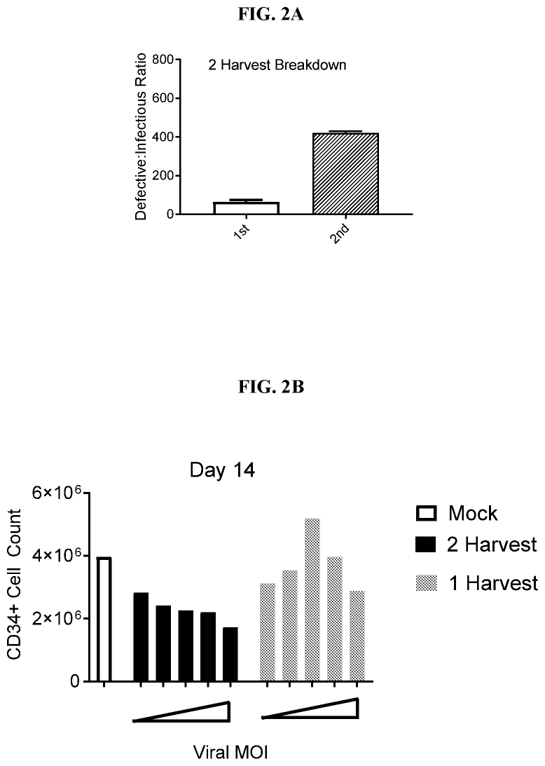

As shown in FIG. 2A, the 2^{nd} Harvest showed a significantly higher defective:infectious ratio as relative to that of 1st Harvest, indicating that the 2^{nd} Harvest contains a large amount of defective lentiviral particles.

Moreover, the lentiviral particles in the 1^{st} Harvest and 2^{nd} Harvest were examined for toxicity to CD34⁺ cells. CD34+ cells were infected with the viral particles and cell toxicity (represented by live cell count) was measured on Day 14. As shown in FIG. 2B, CD34+ cells infected with the 1^{st} Harvest exhibited higher viable cell number as relative to the CD34+ cells infected with the 2^{nd} Harvest. This result indicates that lentiviral particles in the 2^{nd} Harvest are more toxic to CD34+ cells as compared with those in the 1^{st} Harvest. Results of this Example indicate that the single collection approach would be expected to produce lentiviral particles with high amount of infectious virus and less toxic to CD34⁺ cells.

### Example 3: Low Salt Elution in Ion-Exchange Chromatography Yielded Less Defective Lentiviral Particles

This Example compares the infectious ability of lentiviral vectors obtained from ion-exchange chromatography using low salt elution as compared with high salt elution.

Low Salt Elution

The single collection or pooled two collection samples described in Examples 1 and 2 above were filtered through LRF and a 0.45 μM filter and then loaded onto a Mustang Q® filter. Post wash, the bound lentiviral particles were eluted from the Mustang Q filter with 25 mM Tris-HCl, pH 8.0, 600 mM NaCl (Elution Buffer).

High Salt Elution

The single collection or pooled two collection samples described in Examples 1 and 2 above were filtered through LRF and a 0.45 μM filter and then loaded onto a Mustang Q® filter. Post wash, the bound viral particles were eluted with 25 mM Tris-HCl, pH 8.0, 1200 mM NaCl (Elution Buffer).

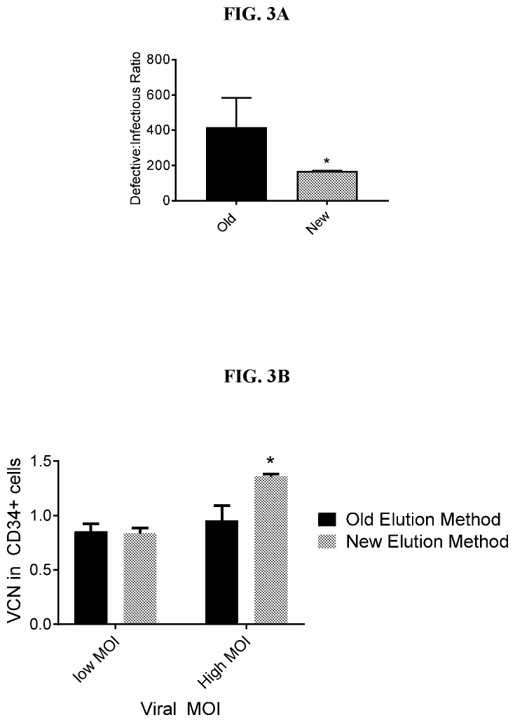

The defective:infectious ratios of viral particles from low salt elution (new) and high salt elution (old) were determined following the methods disclosed in the above examples. The defective:infectious ratio of the lentiviral particles obtained from low salt elution (new) was much lower than that from high salt elution (old). FIG. 3A.

Further, gene transfer efficiency of the lentiviral particles yielded from low salt elution and high salt elution was determined. Normal CD34+ cells were transduced under clinically used transduction conditions. Cells were cultured for 2 weeks and then harvested, DNA extracted and vector copy number VCN determined using qPCR. As shown in FIG. 3B, the lentiviral particles yielded from low salt elution showed higher gene transfer efficiency as relative to those yielded from high salt elution at high MOI; while similar results were observed at low MOI.

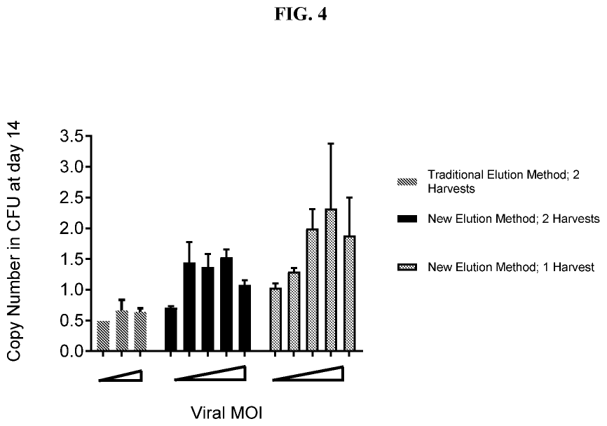

Moreover, transducibility of lentiviral particles yielded from low salt elution of the single collection described above was compared with transducibility of lentiviral particles yielded from low salt elution of pooled two collections and viral particles yielded from high salt elution of single collection. CD34+ cells were transduced with the viral particles from these preparations and VCN of the transgene was measured at day 14. As shown in FIG. 4, best transducibility was observed in viral particles yielded from low salt elution of single collection.

### Example 4: In Vitro and In Vivo Activity of Lentiviral Particles Prepared by Methods Disclosed Herein

Lentiviral Vector Gene Transfer is performed in human CD34⁺ hematopoietic stem and progenitor cells (HSPC), followed by their transplant to recipient immune-deficient mice. It is important to determine the gene transfer into HSPC and their in vivo engraftment potential. The rationale for in vivo validation is because 98-99% of CD34⁺ HSPC are progenitors and only 1-2% are stem cells. Hence in vitro CD34⁺ cell assays are validated in vivo to determining long term engraftability of gene modified cells and stability of vector copy number (VCN) in vivo.

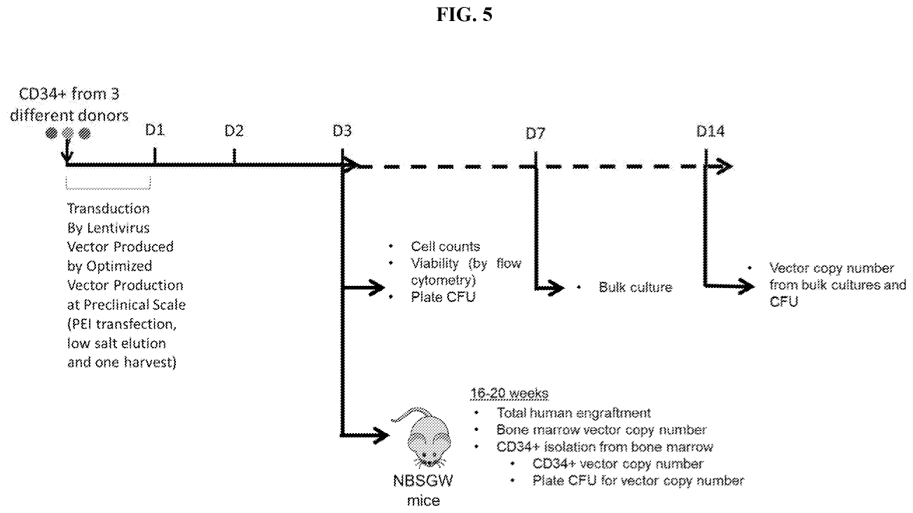

Lentiviral particles prepared by the manufacturing method disclosed herein, involving PEI transfection of lentiviral vectors to host cells, low salt elution for purification of lentiviral particles, and one harvest of lentiviral particles, were examined both in vitro and in vivo gene transfer activities. An exemplary experimental overview is provided in FIG. 5.

Three medium scale gene transfers into 20 million thawed CD34⁺ HSPC from 3 different human donors were performed using the preclinical grade lentiviral particles prepared as disclosed herein at different viral concentrations, following the optimized transduction conditions. Approximately 4×10⁶ CD34⁺ HSPC were transduced increasing vector multiplicity of infection (MOI). CD34+ HSPC were washed after transduction, and then assayed for different assays listed below.

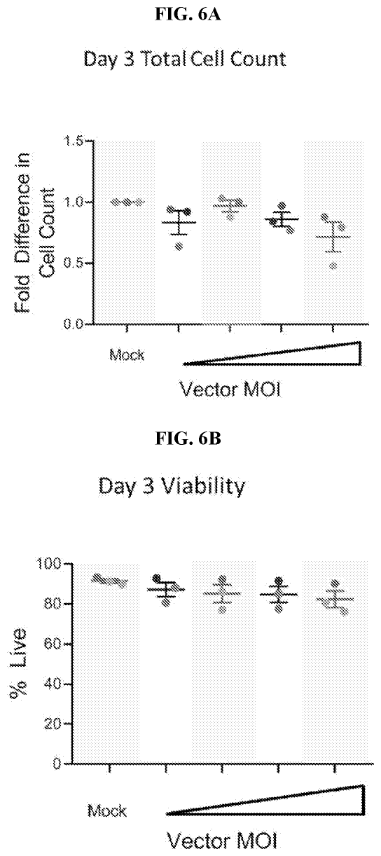

Toxicity of increasing concentration of lentiviral particles (vectors) to CD34⁺ HSPC was studied by assessing CD34⁺ HSPC viable cell numbers in culture (trypan blue exclusion) and live/dead viability flow cytometry analysis of HSPC. The Mock group was used as a control. Total cell count and viability of the HSPCs at Day 3 after transduction are shown in FIG. 6A and FIG. 6B. Total cell count was measured by a hemocytometer and HSPC (and HSC) count was measured by cell count factoring in the percentage of CD34+ cells (and CD34⁺CD38⁻CD90⁺CD45RA⁻ cells) obtained from flow cytometry. Greater than 80% viability was observed with increasing vector MOI, indicating that the lentiviral particles produced by the method disclosed herein did showed negligible toxicity to human CD34⁺ HSPC cells in vitro.

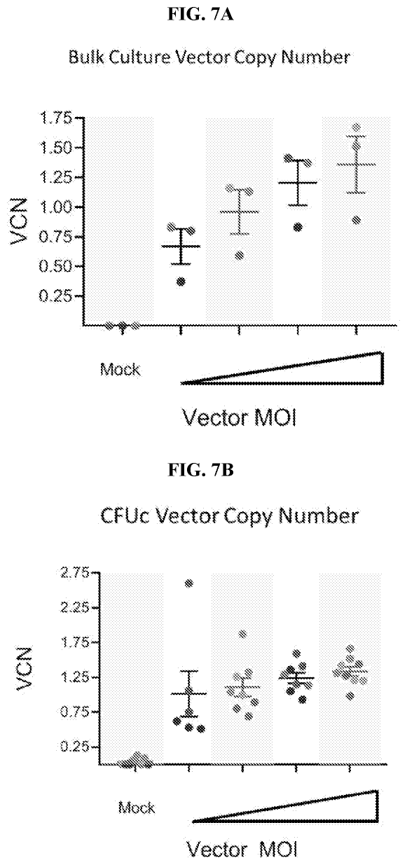

Gene transfer (VCN/cell) was determined in vitro by plating a portion of CD34⁺ HSPC in colony forming assays in triplicate, and pooling colony forming unit cells (CFUc) to determine VCN. A second portion was cultured and expanded in bulk in cytokine rich medium for two weeks and then assessed for VCN. See FIG. 5. The results indicate that the vector copy number/cell (VCN) in CD34⁺ HSPC increased with increasing vector MOI. This validates high quality vector preparation. FIG. 7A and FIG. 7B.

Long term engraftment potential was determined by transplanting the majority of the transduced CD34⁺ HSPC into 3-4 immune deficient mice per experimental group. In the first experiment, 1×10⁶ CD34⁺ HSPC were injected per mouse. For the subsequent two donors, CD34⁺ HSPC were injected in mice in limiting dilution, so that toxicity and engraftability in vivo becomes overt, when limited numbers of HSC are forced to repopulate the mouse hematopoiesis. The recipient mice were followed for 4-5 months. Upon sacrifice, bone marrow from the mice was analyzed for human cell engraftment and VCN. In some instances, human CD34+ cells were isolated from mouse bone marrow, and a portion of CD34+ HSPC were subjected to VCN analysis. See FIG. 5.

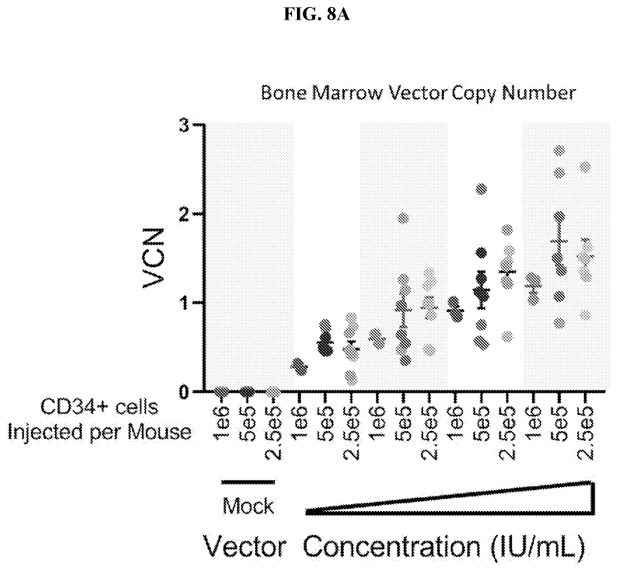

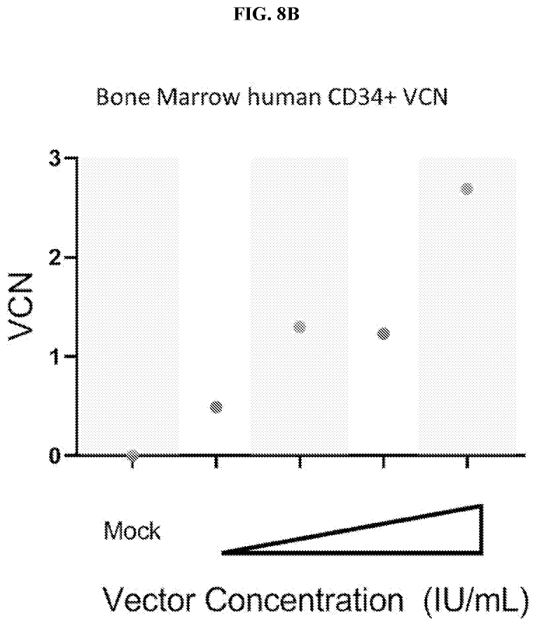

As shown in FIG. 8A and FIG. 8B, the vector copy number derived in vitro was found stable in vivo, including VCN in whole bone marrow and in human CD34⁺ HSPC cells isolated from bone marrow of the recipient mice 4-5 months after the transplant, even when HSPC were injected in limited numbers, and the VCN increased as with increasing vector MOI, similar to the results seen in vitro. These data confirm that there was no toxicity to the long term repopulating stem cells with increasing vector MOI and gene transfer in vitro recapitulated that seen in vivo in animals 4-5 months following transplant from three distinct stem cell donors.

In sum, the results obtained from this study demonstrate that the lentiviral particles prepared by the method disclosed herein, involving PEI transfection, low salt elution, and one harvest, exhibited a number of superior features, including little or no toxicity to host stem cells and stable and high gene transfer efficiency both in vivo and in vitro in HSPC and their progeny, as evidenced by the VCN value in host cells.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method of producing retroviral particles, comprising:
(a) providing a sample containing retroviral particles;
(b) loading the sample onto an ion-exchange column to allow for binding of the retroviral particles to the ion-exchange column; and
(c) eluting the retroviral particle from the ion-exchange column with an elution buffer comprising one or more salts to form a first solution, wherein the elution buffer has a total salt concentration of about 400 mM to 800 mM, and wherein the first solution comprises the retroviral particles.

2. The method of claim 1, wherein the retroviral particle is a lentiviral particle, a gamma retroviral particle, or foamy viral particle.

3. The method of claim 1, wherein the one or more salts comprises calcium chloride, magnesium chloride, sodium chloride, potassium chloride, ammonia chloride, or a combination thereof.

4. The method of claim 1, wherein the total salt concentration of the elution buffer is about 600 mM.

5. The method of claim 1, wherein the sample of step (a) is a culture medium harvested from an in vitro culture of cells transfected with a retroviral vector.

6. The method of claim 5, wherein the culture medium is harvested by a process comprising:
(i) transfecting host cells with the retroviral vector(s);
(ii) culturing the transfected host cells in a first culture medium; and
(iii) harvesting the first culture medium 32-48 hours post transfection.

7. The method of claim 6, wherein step (iii) is performed only once.

8. The method of claim 6, wherein in step (i), the host cells are cultured in a second culture medium, which is replaced with the first culture medium 4-8 hours post transfection prior to step (ii).

9. The method of claim 6, wherein the process further comprises passing the harvested first culture medium through a leukocyte reduction filter, a 0.45 μμ filter, or a combination thereof.

10. The method of claim 5, wherein the retroviral vector carries a gene of interest.

11. The method of claim 10, wherein the gene of interest encodes a gamma-globin protein.

12. The method of claim 11, wherein the gamma-globin protein is a human gamma-globin protein.

13. The method of claim 12, wherein the human gamma-globin protein is a wild-type human gamma-globin protein.

14. The method of claim 13, wherein the wild-type human gamma-globin protein comprises the amino acid sequence of
(SEQ ID NO: 1)
MGHFTEEDKATITSLWGKVNVEDAGGETLGRLLVV
YPWTQRFFDSFGNLSSASAIMGNPKVKAHGKKVLT
SLGDAIKHLDDLKGTFAQLSELHCDKLHVDPENFK
LLGNVLVTVLAIHFGKEFTPEVQASWQKMVTAVAS
ALSSRYH.

15. The method of claim 12, wherein the human gamma-globin protein is a mutated human gamma-globin protein, which comprises a substitution at a position corresponding to position 17 of SEQ ID NO:1.

16. The method of claim 15, wherein the mutated human gamma-globin protein comprises the amino acid sequence of
(SEQ ID NO: 2)
MGHFTEEDKATITSLWDKVNVEDAGGETLGRLLVV
YPWTQRFFDSFGNLSSASAIMGNPKVKAHGKKVLT
SLGDAIKHLDDLKGTFAQLSELHCDKLHVDPENFK
LLGNVLVTVLAIHFGKEFTPEVQASWQKMVTAVAS
ALSSRYH.

17. The method of claim 11, wherein the gene of interest encoding the gamma globin protein comprises one or more intron sequences.

18. The method of claim 5, wherein the retroviral vector is a self-inactivated (SIN) retroviral vector.

19. The method of claim 18, wherein the SIN retroviral vector comprises:
(a) a 5′-LTR region and a 3′-LTR region, wherein the 3′-LTR comprises an upstream polyadenylation (polyA) enhancer signal sequence;
(b) one or more copies of a heterologous poly A signal sequence downstream from the 3′ LTR; and
(c) one or more chromatin insulator elements.

20. The method of claim 19, wherein the upstream polyA enhancer signal sequence is an upstream sequence element (USE) derived from an SV40 late polyA signal sequence.

21. The method of claim 19, wherein the one or more chromatin insulator elements include one or more chicken hypersensitive site-4-elements (cHS4s) or a foamy viral insulator.

22. The method of claim 21, wherein the foamy viral insulator comprises the amino acid sequence of SEQ ID NO:3.

23. The method of claim 19, wherein the retroviral vector further comprises an erythroid lineage specific enhancer element.

24. The method of claim 5, wherein the transfecting step (i) is performed in the presence of polyethylenimine (PEI).

25. The method of claim 5, wherein the transfecting step (i) and the culturing step (ii) are performed in a 10-layer cell stack or in a bioreactor.

26. The method of claim 5, wherein steps (i)-(iii) are performed in the absence of chloroquine.

27. The method of claim 5, wherein the cells are not cultured in a conditioned medium.

28. The method of claim 5, wherein the first culture medium contains about 1% to about −6% fetal bovine serum (FBS).

29. The method of claim 28, wherein the first culture medium contains about 3% FBS.

30. The method of claim 5, wherein the first medium is a serum free medium.

31. The method of claim 1, further comprising subjecting the first solution collected from step (c) to a 1:1 dilution to form a second solution.

32. The method of claim 31, further comprising contacting the second solution with host cells to deliver the retroviral particles to the host cells.

33. The method of claim 32, wherein the host cells are human hematopoietic cells.
